(19) United States
(12) Patent Application Publication
(10) Pub. No.: US 2010/0234938 A1
(43) Pub. Date: Sep. 16, 2010

(54) Grasper System For Placement Of Intraluminal Device

(76) Inventors: Syde A. Taheri, Williamsville, NY (US); Robert LaDuca, Santa Cruz, CA (US)

Correspondence Address: WALTER W. DUFT;LAW OFFICES OF WALTER W. DUFT, 8616 MAIN ST, SUITE 2, WILLIAMSVILLE, NY, 14221, US

(21) Appl. No.: 12/621,247

(22) Filed: Nov. 18, 2009

Related U.S. Application Data
(60) Provisional application No. 61/115,924, filed on Nov. 18, 2008.
(60) Provisional application No. 61/171,350, filed on Apr. 21, 2009.

(51) Int. Cl. A61F 2/84

(52) U.S. Cl. 623/1.23

(57) ABSTRACT

An apparatus for use within a vasculature includes a primary guider having a proximal end, a distal end and a medial portion extending from the proximal end to the distal end. The proximal end includes a proximal opening. The distal end includes a distal opening. The medial portion includes a lumen interconnecting the proximal opening and the distal opening. A grasper is provided at the distal end. A grasper controller is provided for controlling the grasper. The apparatus has various uses, including as a device to deploy a second intraluminal device for attachment to a first intraluminal device within a vasculature. The grasper may be used to grasp the first intraluminal device in order to facilitate attachment of the second intraluminal device. The apparatus may also be used for filtering, with the grasper being constructed to trap blood-borne material while permitting bloodflow.

### CROSS-REFERENCE TO RELATED APPLICATIONS

This application claims benefit of the filing date under 35 U.S.C. 119(e) of the following U.S. Provisional Applications: U.S. Provisional Patent Application No. 61/115,924, filed Nov. 18, 2008, and U.S. Provisional Patent Application No. 61/171,350, filed Apr. 21, 2009. The entire contents of both of said provisional applications are hereby incorporated herein by this reference.

### BACKGROUND OF THE INVENTION

1. Field of the Invention

This invention relates to cardiovascular disease and the treatment thereof. More particularly, the invention pertains to a system and method for placing a graft, stent or other intraluminal device within a vasculature, particularly at a vascular branch junction.

2. Description of the Prior Art

By way of background, grafts, stents and other intraluminal devices have been used for many years to restore functionality to diseased or damaged portions of a vasculature. For example, stented grafts are commonly used to repair aneurysms in large vessels such as the aorta. It is desirable to implant such devices using endovascular techniques to avoid the morbidity and mortality associated with invasive surgical procedures. Percutaneous delivery can often be performed with only local anesthesia and sedation, whereas surgery typically requires general anesthesia, which carries its own risks.

It is sometimes necessary to place an intraluminal device at a branch junction within a patient's vasculature. One example is the region where the abdominal aorta divides into the left and right common iliac arteries. An expandable, generally Y-shaped, bifurcated stent-graft device having a primary limb and two depending branch limbs has been used for abdominal aortic aneurysm (AAA) exclusion in this region. The stent-graft primary limb has an upper end that seats in the abdominal aorta above the aneurysm. The lower end of the primary limb forms a flow dividing graft junction that transitions into an ipsilateral ilac limb and a contralateral limb that respectively seat in the right and left common iliac arteries.

In a modular AAA stent-graft design, the device comprises component parts that are assembled in vivo. A primary main body component comprises the primary limb together with a fully formed ipsilateral iliac limb and a short contralateral pant leg that terminates at a contralateral gate opening. The ipsilateral iliac limb is designed to seat in the right common iliac artery. The contralateral short pant leg aligns with the left common iliac artery but is not long enough reach this artery. Instead, the contralateral gate is designed to receive a contralateral iliac extension limb representing a secondary component of the stent-graft. The extension limb extends from the short pant leg and is long enough to properly seat in the left common iliac artery.

The rationale for the modular design is to reduce the size of the components that must be deployed to the repair site via a sheath introducer. This introducer must be small enough to be inserted into the patient's right femoral artery and advanced through the right common iliac artery to the abdominal aorta. After the main body graft has been deployed in this fashion, the extension limb is similarly deployed using a sheath introducer and a guide wire inserted into the patient's left femoral artery and advanced through the left common iliac artery. The guide wire is used to cannulate the opening of the contralateral gate. The extension limb is advanced a short distance into the short pant leg and attached thereto to provide a full length contralateral iliac limb.

The existing technique for extension limb deployment can be difficult and time consuming, particularly in patients having unfavorable anatomy. In particular, the guide wire sometimes cannot be easily guided into the contralateral gate because the patient has a highly angulated iliac junction or an aneurysm sac that is large relative to the comparatively small opening of the contralateral gate. It is also possible for the contralateral gate to be hidden due to the stent-graft assuming a twisted “ballerina” deployment of the main body graft. A further complication arises from the fact that the guide-wire manipulation can result in atheroemboli and/or thromboemboli being released from the aneurysm site and traveling to the small capillaries of the leg. This can produce acute lower limb ischemia and a condition known as “trash foot.” Excessive extension limb deployment time also means increased radiation exposure for both the patient and the physician.

It is to the deployment of stents, grafts and other intraluminal devices at vascular junctions that the present invention is directed. Of particular interest is the staged deployment of a first intraluminal device followed by a second intraluminal device that attaches to the first intraluminal device.

### SUMMARY OF THE INVENTION

An apparatus for use within a vasculature includes a primary guider having a proximal end, a distal end and a medial portion extending from the proximal end to the distal end. The proximal end includes a proximal opening. The distal end includes a distal opening. The medial portion includes a lumen interconnecting the proximal opening and the distal opening. A grasper is provided at the distal end. A grasper controller is provided for controlling the grasper. The apparatus has various uses, including as a device to deploy a second intraluminal device for attachment to a first intraluminal device within a vasculature. The grasper may be used to grasp the first intraluminal device in order to facilitate attachment of the second intraluminal device. The apparatus may also be used for filtering, with the grasper being constructed to trap blood-borne material while permitting bloodflow.

### BRIEF DESCRIPTION OF THE DRAWINGS

The foregoing and other features and advantages of the invention will be apparent from the following more particular description of example embodiments, as illustrated in the accompanying Drawings, in which:

FIG. 1 is a side view showing an example grasper device for placing an intraluminal device, with the device being in a non-deflected grasping configuration;

FIG. 2 is a side view showing the grasper device in a deflected grasping configuration;

FIG. 3 is a side view showing the grasper in a non-deflected, retracted configuration;

FIG. 4 is a side view showing the grasper in a deflected, retracted configuration;

FIG. 5 is a cross-sectional view showing a handle of the grasper device;

FIG. 6 is a cross-sectional view showing a distal end of the grasper device;

FIG. 7 is a perspective view showing a proximal end of the grasper device;

FIG. 8 is a cross-sectional view showing a sheath dilator that may be used with the grasper device;

FIG. 9 is a cross-sectional view showing a removable guide wire catheter that may be used with the grasper device;

FIG. 10 is a diagrammatic view showing an aorto-iliac stent-graft deployed at an aorto-iliac junction prior to attachment of a short limb extender;

FIG. 11 is a diagrammatic view showing the grasper device grasping the short limb of the stent-graft of FIG. 10 in order to direct a guide wire into the short limb;

FIG. 12 is a diagrammatic view showing a short limb extender being advanced along a guide wire of the grasping device;

FIG. 13 is a diagrammatic view showing the stent-graft of FIG. 10 following attachment of the short limb extender;

FIG. 14 is a side view showing the grasper device of FIG. 1 in a retracted configuration in combination with an example deflectable guide catheter;

FIG. 15 is a side view showing the combination of FIG. 14 with the grasper device in a grasping configuration;

FIG. 16 is a perspective view showing the grasper device mounted to the deflectable guide catheter;

FIG. 17 is a perspective view showing a proximal end of the deflectable guide catheter wherein a deflection controller is adjusted to a first position in which the guide catheter distal end (not shown) is not deflected;

FIG. 18 is a perspective view showing the proximal end of the deflectable guide catheter wherein the deflection controller is adjusted to a second position in which the guide catheter distal end (not shown) is deflected;

FIG. 19 is a further perspective view showing the proximal end of the deflectable guide catheter;

FIG. 20 is a cross-sectional centerline view of the proximal end of the deflectable guide catheter;

FIG. 21 is a perspective view showing a proximal end of a modified deflectable guide catheter wherein a deflection controller according to the modified construction is adjusted to a first position in which the guide catheter distal end (not shown) is not deflected;

FIG. 22 is a perspective view showing the proximal end of the deflectable guide catheter of FIG. 21 wherein the deflection controller is adjusted to a second position in which the guide catheter distal end (not shown) is deflected;

FIG. 23 is a cross-sectional centerline view of the proximal end of the deflectable guide catheter of FIG. 21 wherein the deflection controller is adjusted to a second position in which the guide catheter distal end (not shown) is not deflected;

FIG. 24 is a cross-sectional centerline view of the proximal end of the deflectable guide catheter of FIG. 21 wherein the deflection controller is adjusted to a second position in which the guide catheter distal end (not shown) is deflected;

FIG. 25 is a further perspective view showing the proximal end of the deflectable guide catheter of FIG. 21;

FIG. 26 is a diagrammatic view showing the distal end of the combined grasper device and deflectable guide catheter being controllably deflected while approaching the contralateral short pant leg of a modular abdominal aortic aneurysm stent-graft; and

FIG. 27 is a diagrammatic view showing the distal end of the combined grasper device and deflectable guide catheter with the distal end of the grasper device being extended to grasp the contralateral short pant leg of a modular abdominal aortic aneurysm stent-graft.

### DETAILED DESCRIPTION OF EXAMPLE EMBODIMENTS

### First Embodiment

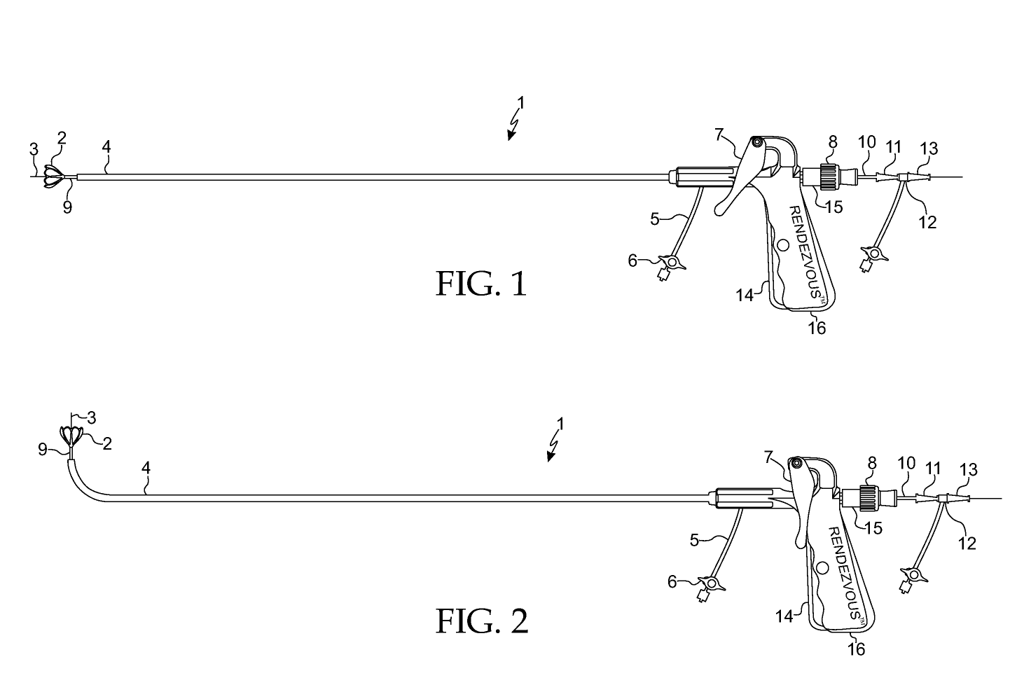

Turning now to FIG. 1, a grasper device 1 is shown that may be used for deploying an intraluminal device, such as a stent or graft, to a location within a vasculature, such as a branch junction. The grasper device 1 is particularly adapted to assist in deploying a second intraluminal device for attachment to a previously deployed first intraluminal device. As described in more detail below, one application for which the grasper device 1 may be used to attach a contralateral iliac limb extension to a contralateral short pant leg of a bifurcated stent-graft deployed at the aorto-iliac junction to repair an abdominal aortic aneurysm (see Background discussion above).

As can be seen in FIG. 1, the grasper device 1 may comprise a handle that may include a handle body 16, an articulating handle component 14, a deflection trigger 7, a plunger assembly 15, a locking hemostasis valve cap 8, a sheath flush port 5 and a sheath flush port stopcock 6. The grasper device 1 also comprises a deflectable sheath 4 whose proximal end is operatively connected to the handle and whose distal end is deflectable to 120 degrees or more by way of a deflection controller that includes the deflection trigger 7. The deflectable sheath 4 may be of any suitable size, such as 5 Fr. to 16 Fr. It may comprise a teflon-lined stainless steel braid reinforced with polyether block amide, polyurethane, or other polymer materials. A hydrophilic coating may be applied to the outside of the deflectable sheath 4 to facilitate movement through a vasculature. The flush port 5 and the flush port stopcock 6 are used to introduce saline solution or other fluids for flushing the inside of the deflectable sheath 4 in order to purge air therefrom prior to use. The stopcock 6 connects to a standard syringe.

The grasper device 1 further comprises a primary guider carried by the deflectable sheath 4 and a secondary guider carried by the primary guider. In the illustrated embodiment, the primary guider comprises an expanded distal tip catheter 26 (see FIG. 6) that includes a catheter shaft having a proximal end shaft 10, a distal end shaft 9 and a medial shaft portion extending from the proximal end to the distal end. The catheter shaft may be formed from any suitable medical grade tubing. If desired, a smooth interior liner could be provided to minimize friction on the secondary guider (see below). The catheter 26 could also be constructed so as to be steerable, like the deflectable sheath 4. In that case, it may be possible to dispense with the deflectable sheath 4 and rely instead on the deflection control capability of the catheter 26 to manipulate the distal end of the grasper device 1 into alignment with an intraluminal device. Alternatively, the deflectable sheath 4 could be retained so that compound deflection capability is provided at the distal end of the grasper device 1. A second embodiment disclosed below illustrates an alternative technique for providing compound deflection capability. Alternatively, if the second embodiment is used, it would also be possible to remove all deflection control capability from the grasper device 1.

The proximal end 10 of the catheter shaft comprises a proximal opening and the distal end 9 of the catheter shaft comprises a distal opening. The proximal end 10 mounts a female luer fitting 11 that receives a Touhy Borst hemostasis valve 12 with a flush port and a stopcock. A standard syringe (or a pump) may be attached to the valve 12 in order to apply a vacuum for removing emboli trapped by the distal tip (see below) of the primary guider. The medial portion of the catheter shaft comprises a lumen interconnecting the proximal opening and the distal opening.

A self-expanding distal tip 2 is attached to the distal end 9 of the catheter shaft. It provides a funnel-shaped grasper having a variable-sized mouth that is adapted to grasp and thereby engage an intraluminal device that has been deployed within a vasculature, such as the short contralateral pant leg of a stent-graft deployed at an aorto-iliac junction (see Background discussion above). The distal tip 2 may be formed from a series of nitinol (or other super-elastic) frame elements attached to a polymer mesh with pore sizes between 25 microns and 200 microns. This allows the distal tip 2 to trap emboli that may be generated by operation of the grasper device 1 while passing blood flow. Any suitable polymer mesh material may be used, including but not limited to nylon, polypropylene, polyethylene, PTFE, etc. A heparin coating may be used to minimize clotting. As an additional design feature, the mesh material may be formed with a series ridges that extend transversely around the walls of the distal tip 2. These ridges may be created by deforming the frame elements in the desired manner. The ridges are designed to help direct the guide wire of the secondary guider (see below) away from the distal tip walls as it is extended into an intraluminal device to which the distal tip 2 has been attached.

The frame elements are connected at their base ends to the distal end 9 of the catheter shaft and are biased so that their free ends expand outwardly when not restrained within the deflectable sheath 4. Alternatively, the frame elements could be restrained in other ways if the deflectable sheath 4 is not present. The nitinol elements may be sized to expand from 5 mm to 25 mm diameter (at their free ends) with a length of between 1 cm and 4 cm. If desired, the distal tip 2 may incorporate a radiopaque material for floroscopic visualization. The radiopaque material may be platinum iridium, tantalum, tungsten, or other, and may be attached as a marker band, or painted onto surface of the distal tip mesh portion. A grasper controller that includes the articulating handle-component 14 of the handle (see below) controls the grasper to engulf and then engage the first intraluminal device. It will be appreciated that other designs may be used to provide a grasper, and that the illustrated design is just one example.

In the illustrated embodiment, the secondary guider is provided by the guide wire 3, which is slidably disposed in the lumen of the primary guider's catheter shaft. The guide wire 3 can be formed from any suitable material, such as 0.035 inch standard guide wire. To facilitate sliding within the catheter shaft, the guide wire 3 may be optionally sheathed within a removable guide wire catheter (see discussion of FIG. 9 below). The secondary guider has a proximal end accessible at the proximal end 10 of the primary guider catheter shaft. A proximal luer fitting 13 is provided at the proximal end of the secondary guider. It attaches to the Touhy Borst hemostasis valve 12. The secondary guider has a distal tip end that can be slideably manipulated (from the proximal end of the secondary guider) to extend from the distal end of the primary guider's catheter shaft. The secondary guider is adapted for advancement into an intraluminal device while the latter is engaged by the distal tip 2 of the primary guider. It serves as a guideway for guiding the second intraluminal device into engagement with the first intraluminal device.

It will be appreciated that in lieu of the secondary guider, it may also be possible to use other guideway designs. For example, the lumen of the primary guider's catheter shaft could potentially be used as a guide for advancing a second intraluminal device to a previously deployed first intraluminal device. This may require enlargement of the catheter shaft, which in turn may require dispensing with the deflectable sheath 4. Alternatively, it may be possible to mount the expandable distal tip 2 to the deflectable sheath 4, which is of sufficient size to accommodate the second intraluminal device.

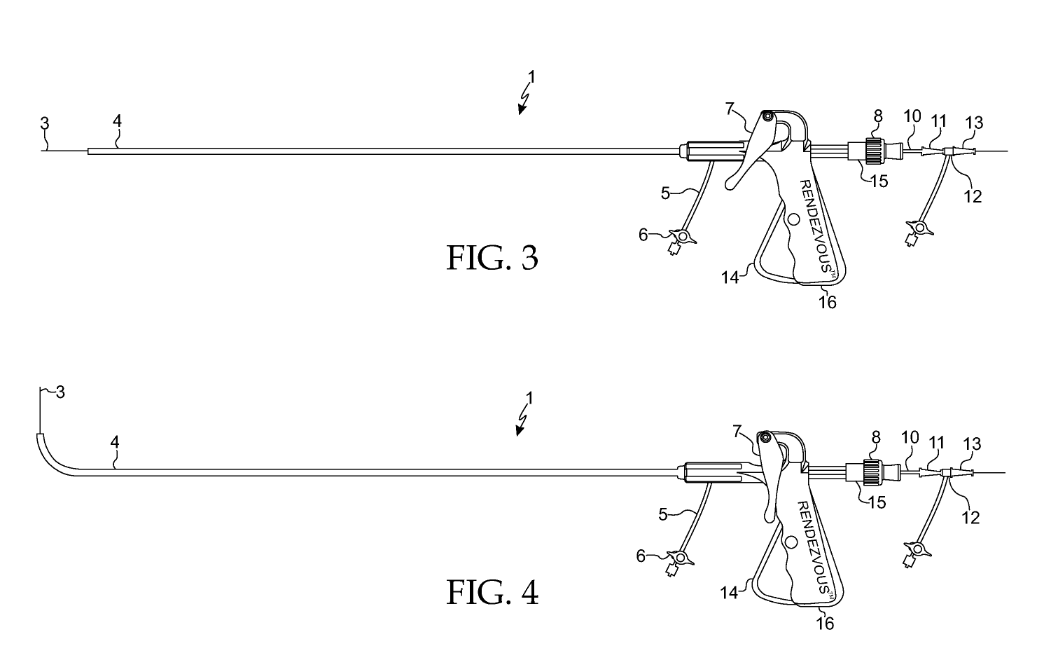

As can be seen in FIGS. 2-4, the grasper device 1 can be controlled at the handle to deflect the distal end of the deflectable sheath 4 (FIGS. 2 and 4) in order to steer the sheath through a vasculature and position the distal tip 2 for engagement with an intraluminal device. The grasper device 1 can also be controlled to extend (FIGS. 1 and 2) and retract (FIGS. 3 and 4) the distal tip 2 from the distal end of the deflectable sheath 4. This allows the distal tip 2 to be operated as a grasper to grasp the outside of an intraluminal device within a vasculature. The size of the distal tip 2 is determined by how far it is extended from the deflectable sheath 4.

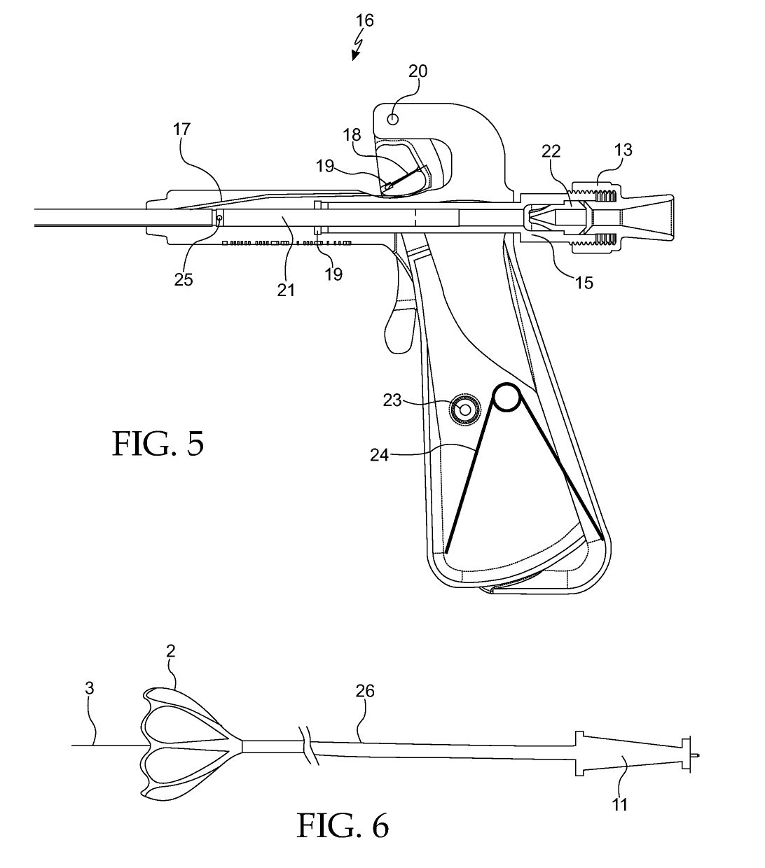

The components of the grasper device 1 that are responsible for the above-mentioned control operations are additionally shown in FIGS. 5 and 6. Deflection control for the deflectable sheath 4 is provided by a deflection controller that includes the trigger 7 (see FIG. 1). As further shown in FIG. 5, the trigger 7 is pivotally mounted to the handle body 16 by way of a stainless steel trigger pivot pin 20. The trigger 7 carries a cable crimp 19 that secures one end of a deflection cable 18. The deflection cable 18 may be provided by a 19-strand, 0.12 inch diameter stainless steel cable, or other suitable material. The deflection cable 18 extends from the cable crimp 19 through an internal path 17 of the handle body 16 to the deflectable sheath 4. The deflection cable 18 feeds through a longitudinal passageway (not shown) that is formed in the wall of the deflectable sheath 14, and which extends the entire length of the deflectable sheath to its distal tip. A pliant tip approximately two inches long at the distal end of the primary guider's catheter shaft can be made more flexible than the remainder of the catheter to facilitate tip deflection when the deflection trigger 7 is activated. Similarly, the deflection cable 18 may be less stiff at its distal end than the remainder of the cable. The same holds for the deflectable sheath 4. These design features impart differential stiffness to the grasper device 1 so that it can be readily steered through a vasculature and so that the distal tip 2 may be positioned for engagement with an intraluminal device. It will be appreciated that other types of mechanism may be used as a deflection controller, and that the illustrated design is just one example.

Control of the distal tip 2 is provided by a grasper controller that includes the articulating handle component 14 of the handle, which is pivotally mounted to the handle body 16 by way of a trigger pivot pin 23. A torsion spring 24 biases the handle component 14 to a home position wherein the distal tip 2 is retracted within the deflectable sheath 4. The handle component 14 connects to a stainless steel plunger hypotube 21 that is bonded or otherwise attached to the plunger assembly 15. The plunger assembly 15 is slideably mounted on the handle body 16. It mounts the locking threaded hemostasis valve cap 8, which in turn squeezes a pinch valve hemostasis gasket seal 22 that clamps to the distal end 10 of the catheter shaft (not shown in FIG. 5). A plunger O-ring seal 19 seals the plunger hypotube 21. When the handle component 14 is squeezed, it pivots on the pivot pin 23 so as to slide the plunger assembly 15 in a distal direction (to the left in FIG. 5). This causes the distal tip 2 to extend out of the deflectable sheath 4 and self-expand. Note that FIG. 5 shows the grasper controller in the distal tip extension position. FIG. 5 also shows a hole 25 within the handle body 16. The hole 25 connects to the sheath flush port 5 of FIG. 1 and allows fluid to be flushed into the deflectable sheath 4. It will be appreciated that other types of grasper controllers may be used in lieu of the above-described components, and that the illustrated design is just one example.

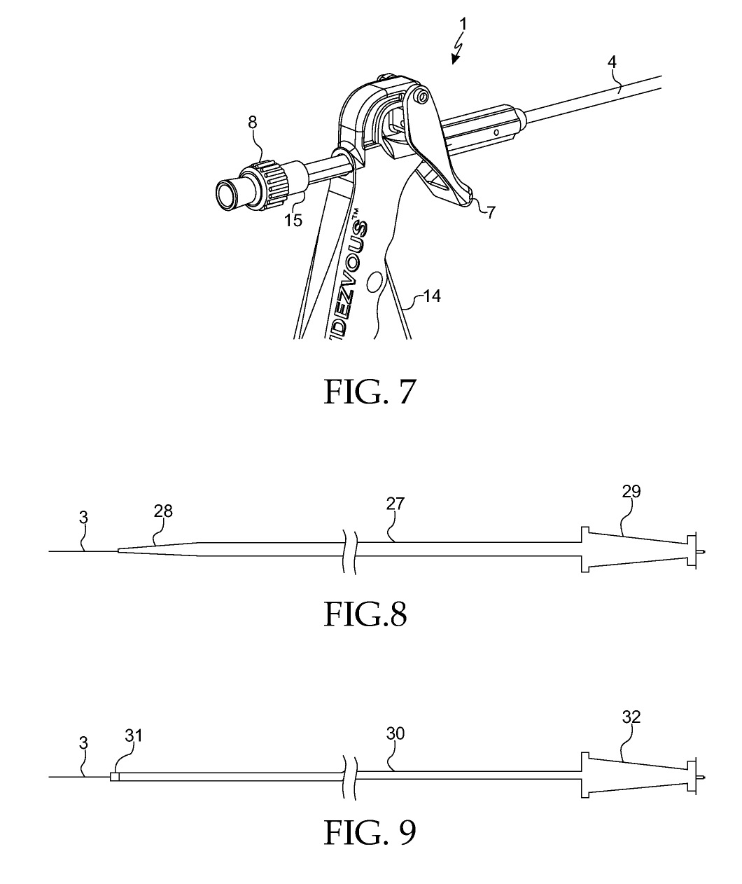

With additional reference now to FIG. 7, it will be seen that the locking threaded hemostasis valve cap 8 includes a funnel end. This makes it easier to insert the distal tip 2 into the grasper device 1 when it is desired to use the primary guider. Advantageously, the primary guider, including the distal tip 2 and the catheter shaft, may be easily removed from the grasper device 1 by loosening the locking threaded hemostasis valve cap 8. This allows the grasper device 1 to be used as a general introducer for endovascular surgery.

For example, as shown in FIG. 8, the primary guider may be removed and a sheath dilator 27 may be inserted in its place to aid in feeding the deflectable sheath 4 through a patient's vasculature. This may be optionally used at the physician's preference and/or in situations requiring a more tractable delivery through unfavorable anatomy such as a tortous illiac artery. The proximal end of the sheath dilator 27 may include a sheath dilator female luer fitting 29. The distal end of the sheath dilator 27 may be formed with a tapered tip 28. The taper length depends upon the diameter of the deflectable sheath 4. It is designed to have a smooth stiffness transition and to accommodate the guide wire 3 with a diameter of 0.035.″ The guidewire 3 extends through a single lumen of the sheath dilator 27 that terminates at the sheath dilator female luer fitting 29. The sheath dilator 27 can be made from a radiopaque polymer such as BASO4 loaded to 20-40% by volume in polyethylene or polypropylene.

As shown in FIG. 9, a removable guide wire catheter 30 may be used to facilitate sliding of the guide wire 3 within the lumen of the primary guider's catheter shaft. The removable guide wire catheter 30 is a translating and removable catheter designed to provide an unobstructed lumen for the guide wire 3 to pass freely through the primary guider of the grasper device 1 regardless of whether the distal tip 2 is deployed from the distal end of the deflectable sheath 4 or is withdrawn into the deflectable sheath.

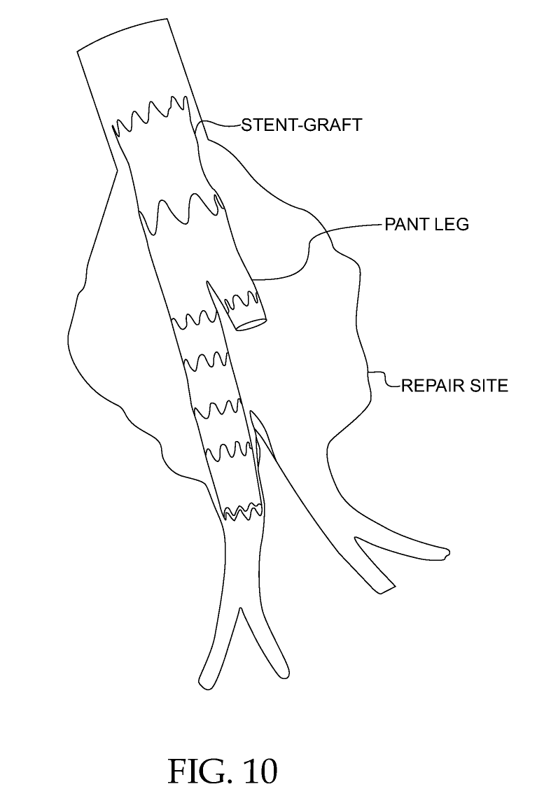

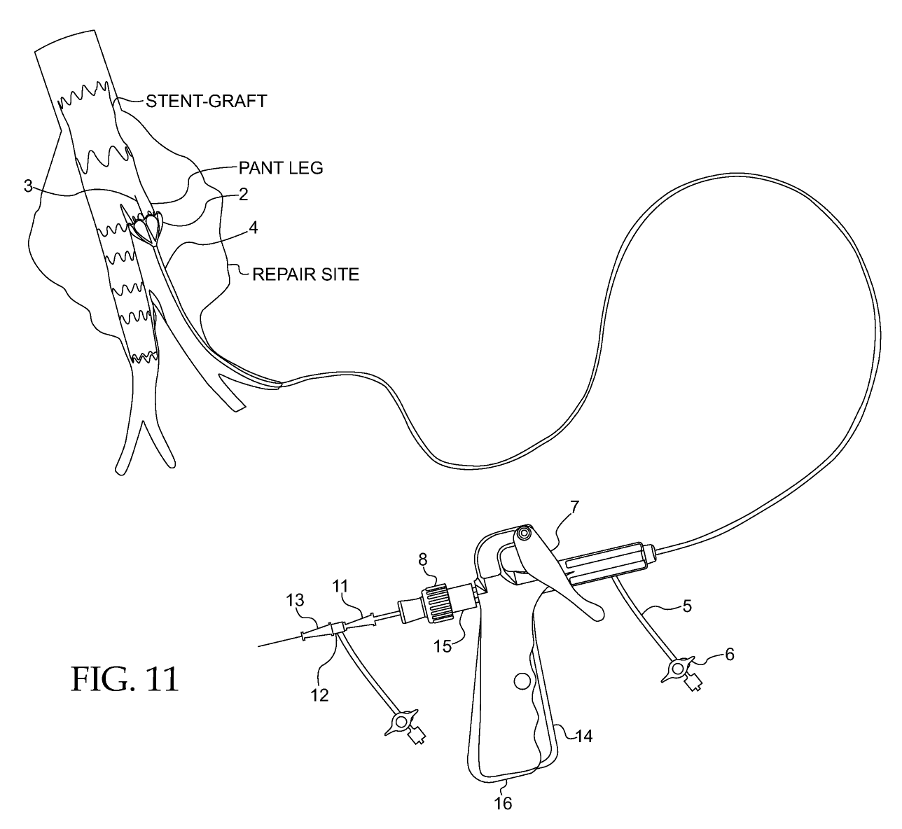

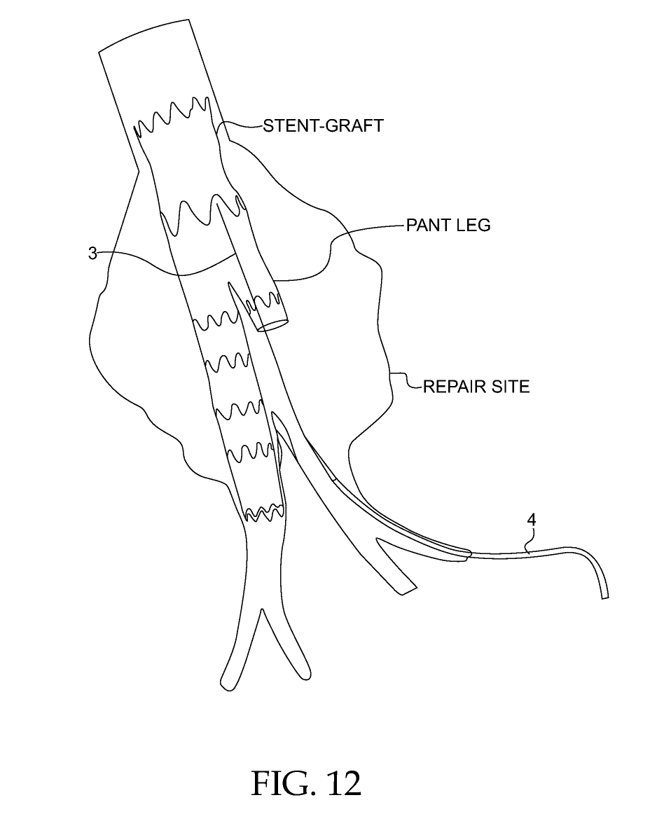

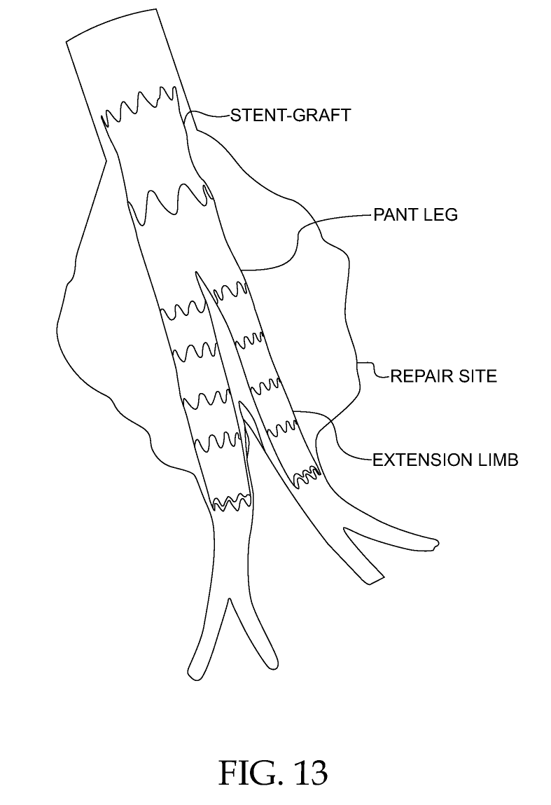

FIGS. 10-13 illustrate an example procedure wherein the grasper device 1 is used to attach an extender to the short contralateral pant leg of a modular, bifurcated, aorto-iliac stent-graft, as shown. FIG. 10 shows the stent graft following initial deployment as described in the Background discussion above. FIG. 11 shows the grasper device 1 after it has been advanced through the left common iliac artery to the repair site. By steering the deflectable sheath 4 and manipulating the distal tip 2, the latter component can be expanded to engulf the opening of the contralateral gate and then be contracted to grasp the short contralateral pant leg. The guide wire 3 may then be advanced into the interior of the contralateral gate. Advantageously, the distal tip 2 is designed to trap emboli that may be generated during the grasper device insertion procedure. These emboli may be removed (e.g., aspirated) after the distal tip 2 has been attached to the short contralateral pant leg by removing the guide wire 3 from the catheter shaft and applying a vacuum to the stopcock of the Touhy Borst hemostasis valve 12. In addition, to prevent emboli from migrating into the right common iliac artery, that artery can be temporarily blocked during distal tip deployment using a balloon catheter inserted into the right femoral artery and advanced to the repair site. As shown in FIG. 12, once the guide wire 3 disposed within the contralateral gate, the grasping device 1 may be removed from the guide wire. The contralateral iliac extension limb may then be advanced over the guide wire to the repair site using a conventional introducer. Alternatively, it is also possible that only the primary guider will need to be removed from the guide wire 3 while leaving the remainder of the grasper device 1 in place. In that case, the deflectable sheath 4 of the grasper device 1 can be used as a general introducer to advance the extension limb. FIG. 13 shows the completion of the modular stent-graft after the contralateral iliac extension limb has been mounted to the short contralateral pant leg.

### Second Embodiment

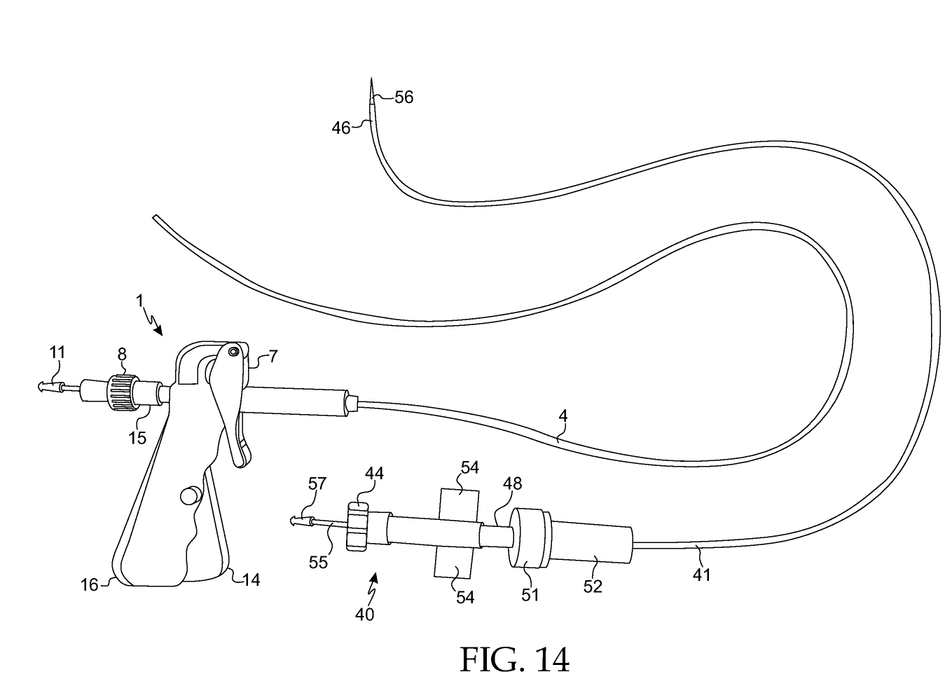

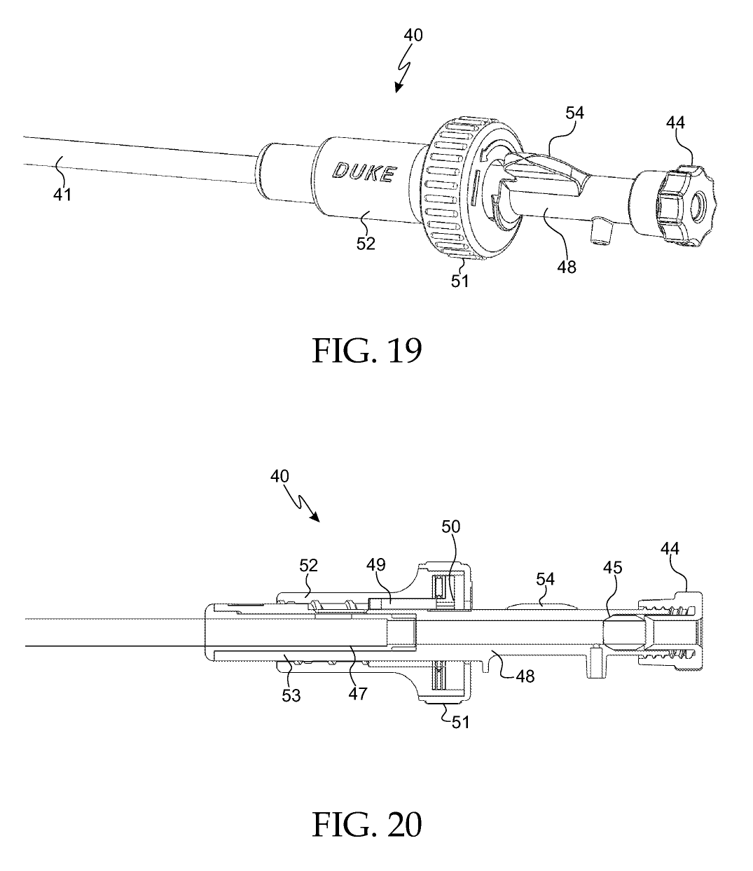

Turning now to FIGS. 14-20, the grasper device 1 of the first embodiment is shown in combination with a deflectable guide catheter 40 to provide additional maneuverability when the deflection components of the first embodiment are designed for single axis deflection only. As will be seen, the second embodiment represents a combination of a first inner assembly and a second outer assembly arranged in a telescoping concentric relationship, with the grasper device 1 representing one example of an inner assembly and the deflectable guide catheter 40 representing one example of an outer assembly. The deflectable guide catheter 40 is particularly adapted to assist in maneuvering the distal tip 2 of the grasper device 1 by facilitating deflection thereof along a second deflection axis that may vary from 0-360 degrees from the first axis of deflection. Alternatively, if the grasper device 1 is constructed without its own deflection capability, the deflectable guide catheter 40 may be used to provide such capability. Again, one application for which the grasper device 1 and the deflectable guide catheter 40 may be used is the attachment of a contralateral iliac extension limb extender to the short contralateral pant leg of a bifurcated stent-graft deployed at the aorto-iliac junction to repair an abdominal aortic aneurysm (see Background discussion above).

In the embodiment shown in the FIGS. 14-20, the grasper device 1 may be constructed in the manner previously described above. As can be seen in FIGS. 14-20, the deflectable guide catheter 40 may comprise a deflectable sheath 41, a sheath flush port 42 and a sheath flush port stopcock 43. The deflectable sheath 41 includes a free distal end 46 and a proximal end 47 that is operatively connected to a deflection controller body 48. The deflectable sheath distal end 46 is deflectable to 120 degrees or more. The deflectable sheath 41 may be of any suitable size, such as 5 Fr. to 16 Fr. or larger, so that it is large enough to receive the deflectable sheath 4 of the grasper device 1. The deflectable sheath 41 may comprise a teflon-lined stainless steel braid reinforced with polyether block amide, polyurethane, or other polymer materials. A hydrophilic coating may be applied to the outside of the deflectable sheath 41 to facilitate movement through a vasculature.

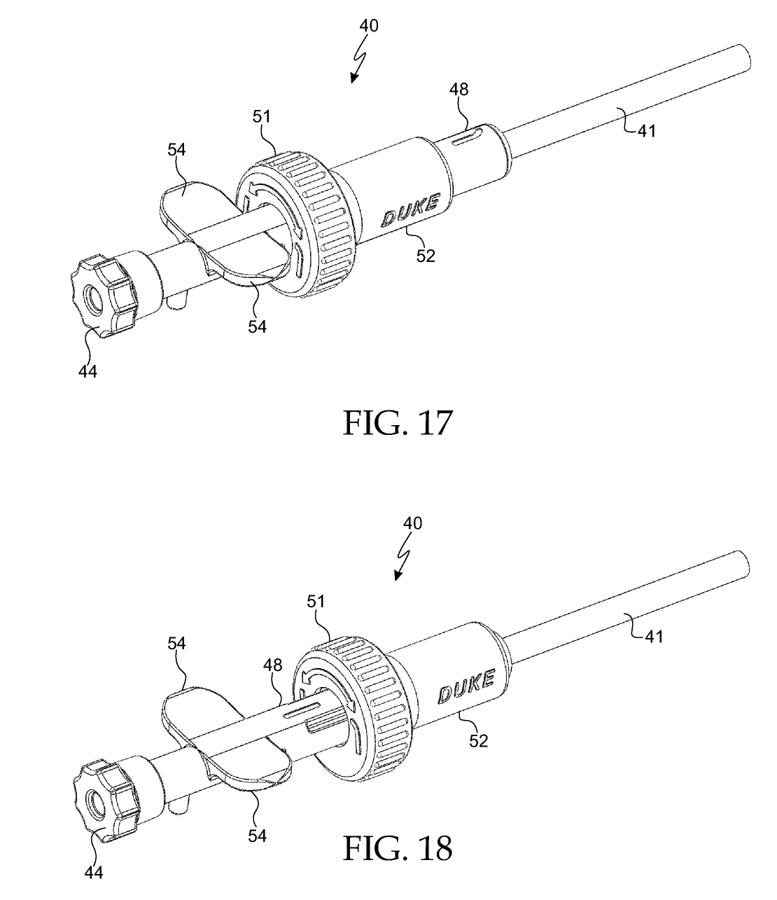

A deflection cable (not shown) may be provided by a 19-strand, 0.12 inch diameter stainless steel cable, or other suitable material. The proximal end of the deflection cable extends through an internal path 49 of the deflection controller body 48 and includes a cable crimp (not shown) that attaches to a deflection cable anchor 50 that is slidable along the outside of the deflection controller body. The deflection cable anchor 50 is engaged by a rotatable deflection control knob 51 formed at the proximal end of a threaded control knob housing 52. The threaded control knob housing 52 is threaded to a thread rack 53 formed on the deflection controller body 48. Rotation of the rotatable deflection control knob 51 rotates the inter-engaging threads, causing the deflection cable anchor to translate along the deflection controller body 48. FIGS. 17 and 18 illustrate this translation motion.

The deflection cable feeds distally from the deflection controller body 48 through a longitudinal passageway (not shown) that is formed in the wall of the deflectable sheath 41. This passageway extends the entire length of the deflectable sheath to its distal tip. As the rotatable deflection control knob 51 is rotated (e.g., clockwise when viewed from the vantage point of the physician), translation of the threaded deflection control knob housing in a proximal direction will put tension on the deflection cable, causing the distal end 46 of the deflectable sheath 41 to deflect. Rotation of the rotatable deflection control knob 51 in the opposite direction rotated (e.g., counterclockwise when viewed from the vantage point of the physician) will cause the distal end 46 to straighten. Torque control wings 54 may be provided to provide finger leverage while rotating the rotatable deflection control knob 51.

A pliant tip approximately two inches long at the distal end 46 of the deflectable sheath 41 can be made more flexible than the remainder of the sheath to facilitate tip deflection when the deflection trigger 7 is activated. Similarly, the deflection cable may be less stiff at its distal end than the remainder of the cable. These design features impart differential stiffness to the deflectable sheath 41 so that it can be readily steered through a vasculature and be positioned relative to an intraluminal device. It will be appreciated that other types of mechanism may be used as a deflection controller, and that the illustrated design is just one example.

The flush port 42 and the flush port stopcock 43 are used to introduce saline solution or other fluids for flushing the inside of the deflectable sheath 41 in order to purge air therefrom prior to use. The stopcock 43 connects to a standard syringe.

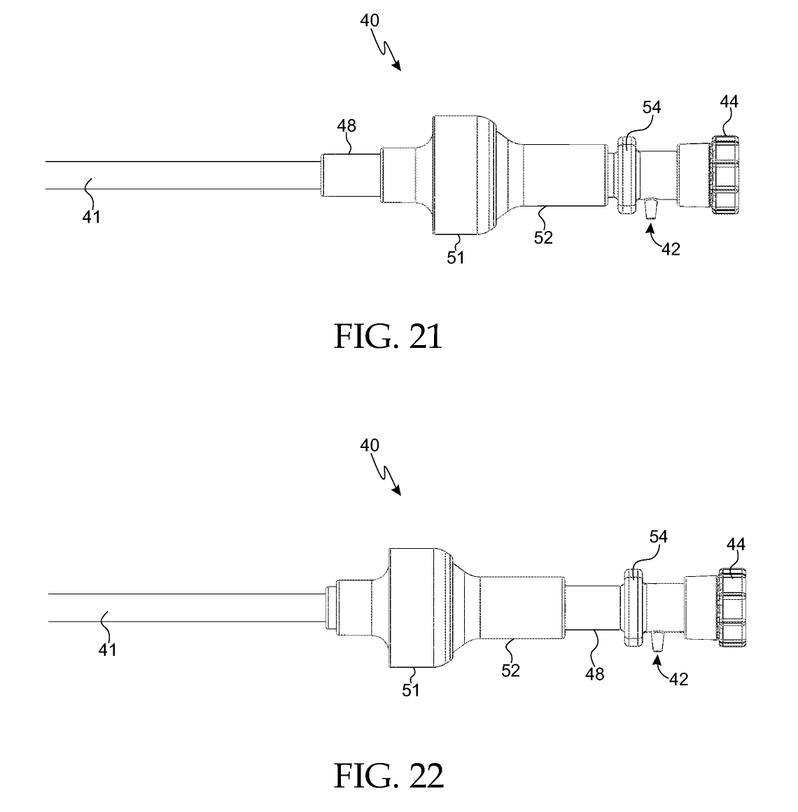

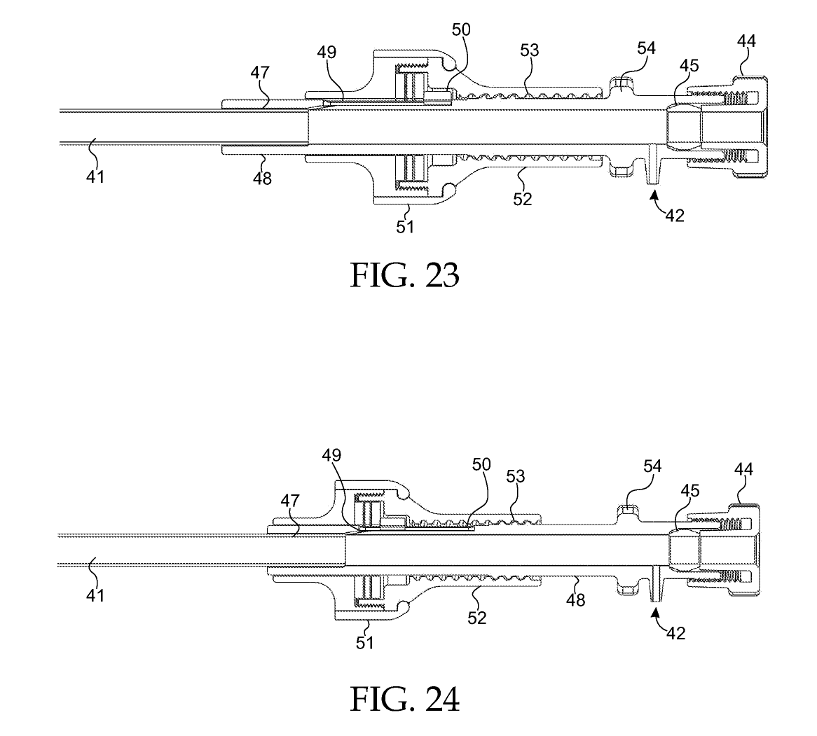

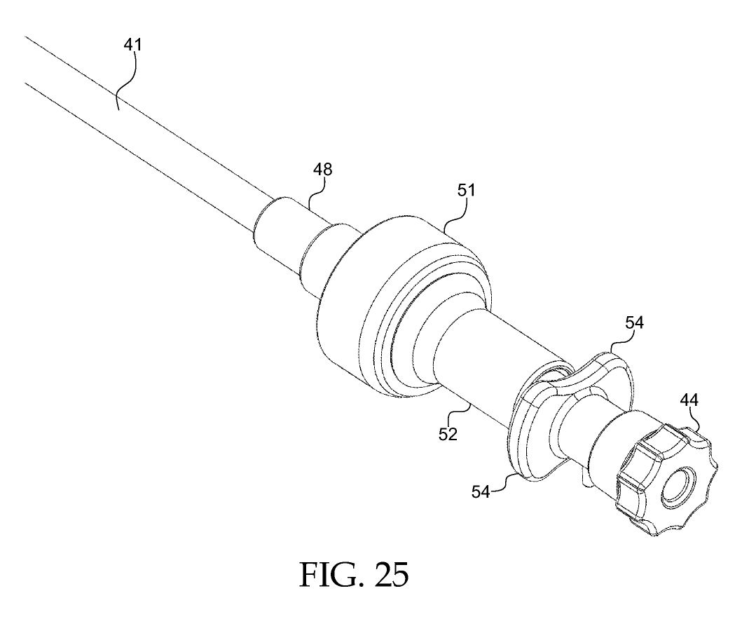

FIGS. 21-25 illustrate a modified construction of the deflectable guide catheter 40. The principal difference is that the torque control wings 54 are smaller and the rotatable deflection control knob 51 includes a rubber finger grip instead of knurling.

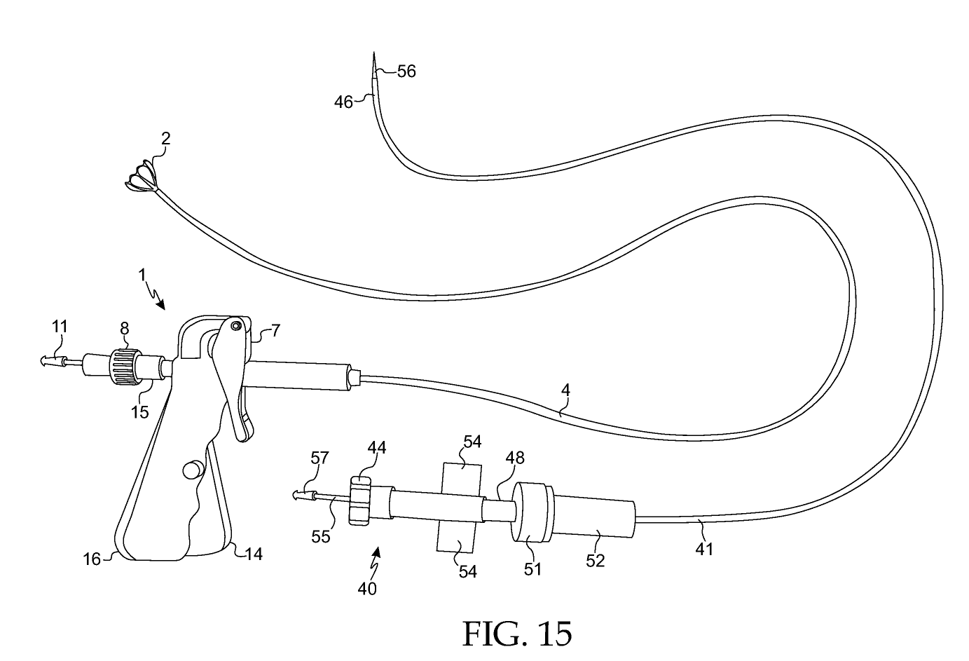

The deflectable guide catheter 41 is designed to be advanced through a vasculature prior to the grasper device 1. As shown in FIGS. 14-15, a sheath dilator 55 may be fed through the deflectable sheath 41 to aid in feeding the deflectable guide catheter 40 through a patient's vasculature. This may be optionally used at the physician's preference and/or in situations requiring a more tractable delivery through unfavorable anatomy such as a tortuous iliac artery. The distal end of the sheath dilator 55 may be formed with a tapered tip 56. The taper length depends upon the diameter of the deflectable sheath 41. It is designed to have a smooth stiffness transition. The proximal end of the sheath dilator 55 may include a sheath dilator female luer fitting 57. The sheath dilator 55 can be made from a radiopaque polymer such as BASO4 loaded to 20-40% by volume in polyethylene or polypropylene.

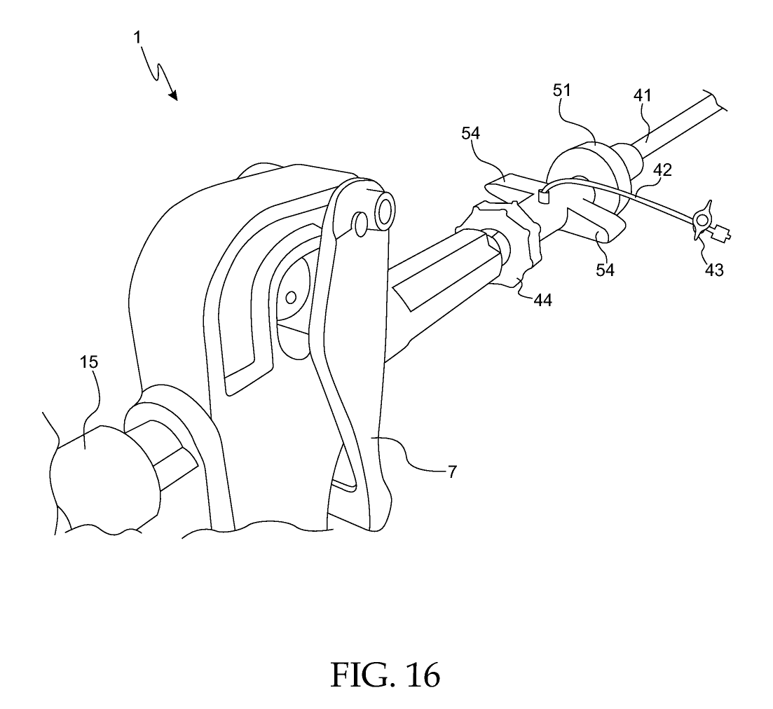

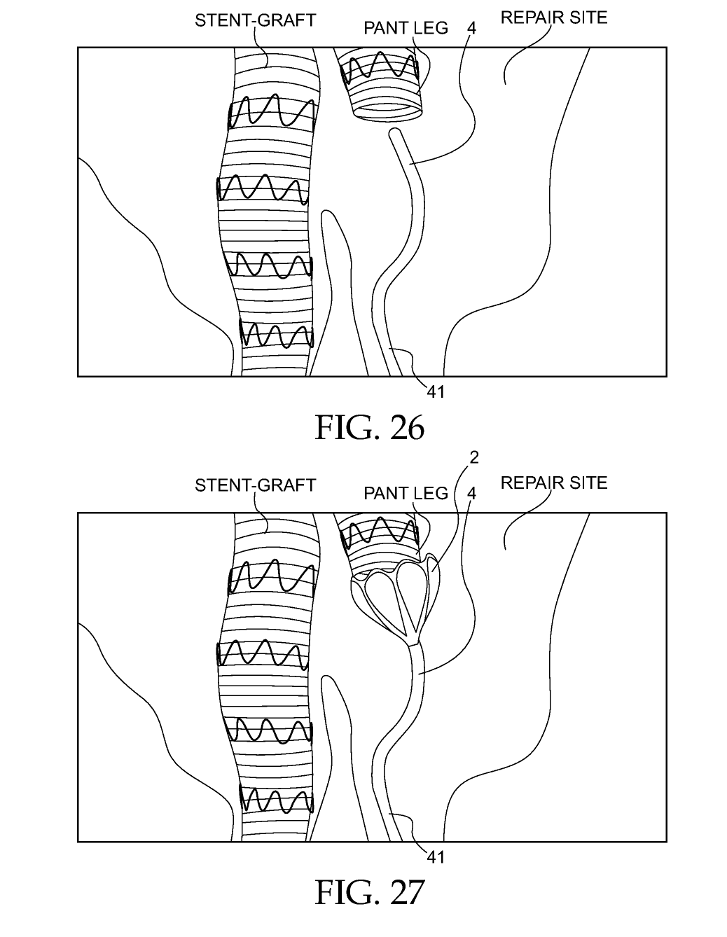

Once the deflectable guide catheter 41 has been deployed to an implantation site, the sheath dilator 55 may be removed from the deflectable sheath 41. The deflectable sheath 4 of the grasper device 1 may be fed through the deflectable sheath 41 and the locking threaded hemostasis valve 44 may be clamped down to secure the grasper device to the deflectable guide catheter 41 in a concentric relationship. This configuration is shown in FIG. 16. Alternatively, deployment of the deflectable guide catheter 41 could be performed with the grasper device 1 already mounted thereto. In that case, the sheath dilator 27 of the grasper device 1 could be used for navigating the vasculature. With the distal end of the combined assembly in the vicinity of the target implantation site, compound deflection maneuvering may be achieved by selectively controlling the rotatable deflection control knob 51 of the deflectable guide catheter 41 and the deflection trigger 7 of the grasper device 1. In addition, the hemostasis valve 44 may be loosened so that the grasper device 1 may be telescoped relative to the deflectable guide catheter 41 to achieve a further range of motion. FIGS. 26-27 are illustrative. In both figures, the deflectable sheath 41 has been deflected in a first direction to lead it away from the aortic wall while the deflectable sheath 4 has been telescoped from the deflectable sheath 41 and deflected in a second direction to line up with the contralateral gate. As shown in FIG. 27, the distal tip 2 of the grasper device 1 may then be deployed to engage the short contralateral pant leg.

As previously mentioned, the deflectable guide catheter 40 could be used in lieu of providing a steering system in the grasper device 1. In that case, the deflectable sheath 4, the steering trigger 7 and other steering components could be eliminated from the grasper device 1. Instead, the expanded distal tip catheter 26 would be carried directly in the deflectable sheath 41 of the deflectable guide catheter.

Accordingly, an apparatus and method for placement of an intraluminal device within a vasculature has been disclosed. Intraluminal devices that can be deployed in this manner include stented grafts for the aorto-iliac junction, as described above. Other devices include but are not limited to aortic arch grafts having branches that extend into one or more of the brachiocephalic artery, the left common carotid artery and the left subclavian artery.

Although example embodiments of the inventions have been shown and described, it should be apparent that many variations and alternative embodiments could be implemented in accordance with the teachings herein. It is understood, therefore, that the invention is not to be in any way limited except in accordance with the spirit of the appended claims and their equivalents.

What is claimed is:

1. An apparatus for use within a vasculature, comprising:
primary guider having a proximal end, a distal end and a medial portion extending from said proximal end to said distal end;
said proximal end comprising a proximal opening;
said distal end comprising a distal opening;
said medial portion comprising a lumen interconnecting said proximal opening and said distal opening;
a grasper at said distal end; and
a grasper controller for controlling said grasper.

2. The apparatus of claim 1, further including:
a secondary guider slidably disposed in said primary guider lumen;
said secondary guider having a proximal end accessible at said proximal end of said primary guider;
said secondary guider having a distal end adapted to extend from said distal end of said primary guider;
said secondary guider being adapted for advancement into a first intraluminal device when said first intraluminal device is engaged by said grasper; and
said secondary guider being further adapted to guide a second intraluminal device to said first intraluminal device for attachment thereto.

3. The apparatus of claim 1, wherein said grasper comprises a variable size mouth on said primary guider defining said distal opening, and wherein said grasper controller is adapted to vary said mouth to manipulate said distal opening between a first enlarged configuration of relative large size and a second narrowed configuration of relatively small size.

4. The apparatus of claim 3, wherein said grasper controller comprises a sheath disposed on the outside of said grasper, said grasper being slidable between an extended position wherein said grasper is extended from said sheath and said mouth is in said enlarged configuration and a retracted position wherein said grasper is retracted in said sheath and said mouth is closed by said sheath to said narrowed configuration.

5. The apparatus of claim 4, wherein said sheath has a proximal end mounted to a handle and a distal end adapted to cover all or a portion of said distal end of said primary guider when said sheath is in said retracted position.

6. The apparatus of claim 5, wherein said sheath is controllably deflectable and said apparatus comprises a sheath deflection controller adapted to deflect a distal end of said sheath for positioning said grasper in said vasculature.

7. The apparatus of claim 2, wherein said grasper comprises a mouth being sized to engulf an outside portion of said first intraluminal device, and wherein said grasper controller is adapted to manipulate said grasper to engulf said outside portion.

8. The apparatus of claim 2, wherein said secondary guider comprises one of a guide wire or a secondary guider lumen and a guide wire disposed in said secondary guider lumen.

9. The apparatus of claim 1, wherein said grasper is adapted to trap emboli for removal through said primary guider lumen while passing blood flow.

10. The apparatus of claim 2, wherein said grasper comprises ridges to direct said secondary guider.

11. The apparatus of claim 2, wherein said first intraluminal device comprises one of a bifurcated abdominal aortic aneurysm graft or an aortic arch graft, and said second intraluminal device comprises an extender for extending a limb on said graft.

12. A method of use of the apparatus of claim 1 to attach a second intraluminal device to a first intraluminal device within a vasculature, comprising:
implanting said first intraluminal device in said vasculature;
deploying said apparatus in said vasculature; and
controlling said apparatus to locate said first intraluminal device for attachment of said second intraluminal device.

13. The method of claim 12, wherein said deploying comprises advancing said apparatus through said vasculature to said first intraluminal device.

14. The method of claim 13, wherein said controlling comprises activating said grasper controller of said apparatus to cause said grasper to engage said first intraluminal device.

15. The method of claim 14, wherein said controlling further comprises advancing said secondary guider of claim 2 into said first intraluminal device.

16. The method of claim 15, further including disengaging said grasper from said first intraluminal device and withdrawing all but said secondary guider from said vasculature.

17. The method of claim 16, further including advancing said second intraluminal device along said secondary guider and attaching it to said first intraluminal device.

18. The method of claim 17, further including withdrawing said secondary guider from said vasculature.

19. An apparatus for use within a vasculature, comprising:
a sheath;
a primary guider carried within said sheath;
a grasper on said primary guider adapted to engage an object in said vasculature;
and
a grasper controller in said handle adapted to control said grasper to engage said object.

20. The apparatus of claim 19, further including:
a secondary guider carried by said primary guider; and
said secondary guider being configured to be manipulated at a proximal end thereof for advancement of a distal end thereof to said object when said object is engaged by said grasper.

21. The apparatus of claim 19, wherein said grasper controller comprises a handle adapted to advance said grasper from a first retracted position wherein said grasper is substantially disposed within said sheath to a second extended position wherein said grasper is extended out of said sheath.

22. The apparatus of claim 21 wherein said sheath is operatively connected to said handle.

23. The apparatus of claim 22 further including a sheath deflection controller adapted to deflect a distal end of said sheath for positioning said grasper in alignment with said object, said sheath deflection controller including a mechanism in said handle.

24. The apparatus of claim 19, wherein said grasper is adapted to engulf and grasp an outside portion of said object.

25. An apparatus for use within a vasculature, comprising:
an assembly having a grasper at a distal end thereof adapted to engage an object in said vasculature;
a grasper controller for controlling said grasper to engage said object; and
a deflection controller adapted to position said grasper for alignment with said object.

26. An apparatus for use within a vasculature, comprising:
a first assembly that includes:
a first sheath;
a grasper slidable in said first sheath and adapted to engage an object in said vasculature; and
a grasper controller adapted to control said grasper to engage said object;
a second assembly that includes:
a second sheath;
said second sheath carrying said first sheath while permitting telescopic sliding thereof; and
one or both of said first and second sheath being deflectable a distal end of said first or second sheath.

27. An apparatus for deploying a second intraluminal device for attachment to a first intraluminal device within a vasculature using compound deflection control, comprising:
a first assembly that includes:
primary guider having a proximal end, a distal end and a medial portion extending from said proximal end to said distal end;
said proximal end comprising a proximal opening;
said distal end comprising a distal opening;
said medial portion comprising a lumen interconnecting said proximal opening and said distal opening;
a grasper at said distal end adapted to engage said first intraluminal device;
a grasper controller for controlling said grasper to engage said first intraluminal device;
a first deflectable sheath carrying said primary guider;
a first sheath deflection controller for deflecting a distal end of said first deflectable sheath in a first direction;
a secondary guider slidably disposed in said lumen;
said secondary guider having a proximal end accessible at said proximal end of said primary guider;
said secondary guider having a distal end adapted to extend from said distal end of said primary guider;
said secondary guider being adapted for advancement into said first intraluminal device when said first intraluminal device is engaged by said grasper; and
said secondary guider being further adapted to guide said second intraluminal device to said first intraluminal device for attachment thereto;
a second assembly that includes:
a second deflectable sheath having a proximal end, a distal end and a medial portion extending from said proximal end to said distal end;
said proximal end comprising a proximal opening;
said distal end comprising a distal opening;
said medial portion comprising a lumen interconnecting said proximal opening and said distal opening;
a second sheath deflection controller for deflecting said distal end of said second deflectable sheath in a second direction; and
said second deflectable sheath carrying said first deflectable sheath while permitting telescopic sliding thereof and compound deflection of said grasper.

28. A method of use of the apparatus of claim 27 to attach a second intraluminal device to a first intraluminal device within a vasculature, comprising:
implanting said first intraluminal device in said vasculature;
advancing said second deflectable sheath through said vasculature until its distal end is positioned proximate to said first intraluminal device;
advancing said first deflectable sheath through said second deflectable sheath until its distal end is proximate to said first intraluminal device;
operating said first sheath deflection controller and said second sheath deflection controller while telescoping said first deflectable sheath beyond said second deflectable sheath as necessary to align said grasper with said first intraluminal device;
grasping said first intraluminal device with said grasper; and
advancing said second intraluminal device via said guideway for attachment to said first intraluminal device.

29. An apparatus for use within a vasculature, comprising:
a deflectable sheath comprising a lumen;
a sheath deflection controller for deflecting a distal end of said deflectable sheath; and
a valve at a proximal end of said deflectable sheath to limit blood loss and allow introduction of another device through said deflectable sheath lumen.

30. The apparatus of claim 29, further including:
a valve controller operative to adjustably actuate said valve; and
a flush port at said proximal end of said deflectable sheath disposed distally from said valve.

31. An apparatus for use within a vasculature to provide compound deflection control, comprising:
an outer deflectable sheath;
an outer sheath deflection controller for deflecting a distal end of said outer deflectable sheath in a first direction; and
said outer deflectable sheath having a lumen sized to carry an inner deflectable sheath that is deflectable in a second direction while permitting telescopic sliding thereof.
